(19) United States
(12) Patent Application Publication
(10) Pub. No.: US 2025/0244248 A1
(43) Pub. Date: Jul. 31, 2025

(54) DETECTION DEVICE

(71) Applicant: Japan Display Inc., Tokyo (JP)

(72) Inventor: Yoshihiro AKAHIRA, Tokyo (JP)

(21) Appl. No.: 19/039,371

(22) Filed: Jan. 28, 2025

(30) Foreign Application Priority Data
Jan. 31, 2024 (JP) 2024-012490

(51) Int. Cl. G01N 21/78; C12M 1/34

(52) U.S. Cl. CPC G01N 21/78; C12M 41/36

(57) ABSTRACT

According to an aspect, a detection device includes: a light emitter provided with multiple types of light sources configured to emit light in different colors from one another; and a light receiver in which a plurality of optical sensors are two-dimensionally arranged. The detection device is configured to perform detection processing. In the detection processing, the multiple types of the light sources are turned on at different times from one another, and detection is performed by the optical sensors each time any one of the light sources is turned on. The light sources are provided such that the light sources to be turned on in the detection processing are changeable depending on an object to be detected that is irradiated with the light from the light emitter.

### CROSS-REFERENCE TO RELATED APPLICATION

This application claims the benefit of priority from Japanese Patent Application No. 2024-012490 filed on Jan. 31, 2024, the entire contents of which are incorporated herein by reference.

### BACKGROUND

### 1. Technical Field

What is disclosed herein relates to a detection device.

### 2. Description of the Related Art

Detection devices are known that detect colonies of bacteria or the like generated in a culture medium using a chromogenic enzyme substrate based on a color change that has occurred in the culture medium (refer to, for example, Japanese Patent Application Laid-open Publication No. 2001-252093).

To detect the color of the culture medium, the culture medium is irradiated with white light, and light reflected from the culture medium is detected so as to be divided into three colors by red, green, and blue (RGB) optical filters and is integrated to obtain RGB color components. In such a method that uses the RGB optical filters, a bottom and a peak of a detection intensity of each of the red (R), green (G), and blue (B) color components tend to appear in an individual wavelength band, but intermediate detection intensities between the bottoms and the peaks of the multiple colors may overlap one another. In the wavelength band in which such overlapping of the intermediate detection intensities occurs, the detection intensity according to the peak of light of a color corresponding to a particular wavelength occurring in that wavelength band is difficult to be distinguished from the detection intensity according to the overlapping of the intermediate detection intensities of the multiple colors. Thus, the accuracy of distinction of colors is difficult to be increased.

For the foregoing reasons, there is a need for a detection device capable of more accurately distinguishing colors.

### SUMMARY

According to an aspect, a detection device includes: a light emitter provided with multiple types of light sources configured to emit light in different colors from one another; and a light receiver in which a plurality of optical sensors are two-dimensionally arranged. The detection device is configured to perform detection processing. In the detection processing, the multiple types of the light sources are turned on at different times from one another, and detection is performed by the optical sensors each time any one of the light sources is turned on. The light sources are provided such that the light sources to be turned on in the detection processing are changeable depending on an object to be detected that is irradiated with the light from the light emitter.

### BRIEF DESCRIPTION OF THE DRAWINGS

FIG. 1 is a block diagram illustrating a main configuration of a detection device;

FIG. 2 is a diagram schematically illustrating detection processing of a culture medium performed using the detection device;

FIG. 3 is a graph illustrating an example of a change in light absorbance between presence and absence of colonies on the culture medium;

FIG. 4 is a table illustrating an example of content of correspondence data;

FIG. 5 is a table illustrating exemplary content of colony detection characteristics data;

FIG. 6 is a timing diagram illustrating the detection processing;

FIG. 7 is a flowchart illustrating processing related to the detection processing;

FIG. 8 is a flowchart illustrating the detection processing; and

FIG. 9 is a schematic view illustrating a configuration example of the detection device when light affected by an object to be detected is light transmitted through the object to be detected.

### DETAILED DESCRIPTION

The following describes an embodiment of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing that has already been discussed is denoted by the same reference numeral through the description and the drawings, and detailed description thereof may not be repeated where appropriate.

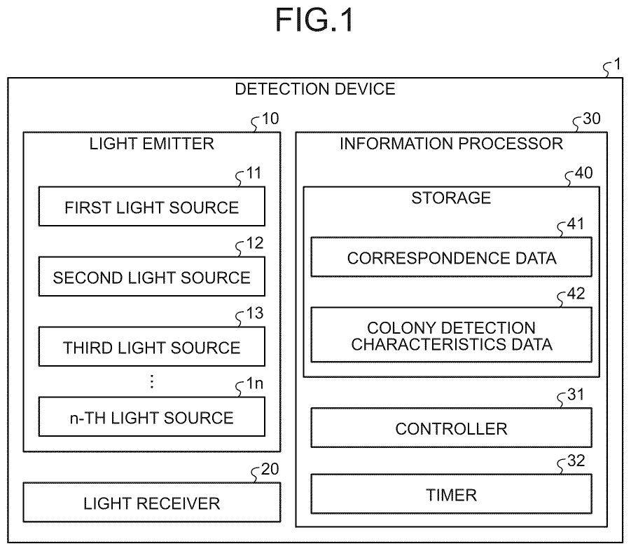

FIG. 1 is a block diagram illustrating a main configuration of a detection device 1. The detection device 1 includes a light emitter 10, a light receiver 20, and an information processor 30.

The light emitter 10 is provided with a plurality of types of light sources that emit light in respective different colors. Thus, the light emitter 10 is provided so as to be capable of emitting the light in the number of colors corresponding to the types of the light sources. FIG. 1 illustrates a first light source 11, a second light source 12, a third light source 13, . . . , an n-th light source In as the multiple types of the light sources. n indicates the number of types of the light sources. Hereinafter, the term “types of the light sources” is synonymous with the number of colors of the light that can be emitted from the light emitter 10. For example, if the number of types of the light sources is four, the number of colors of the light that can be emitted from the light emitter 10 is four. n is a natural number equal to or larger than four.

The first light source 11, the second light source 12, the third light source 13, . . . , the n-th light source 1n are light-emitting elements having respective different emission spectra. Specifically, the first light source 11, the second light source 12, the third light source 13, . . . , the n-th light source In are light-emitting diodes (LEDs), for example. When the first light source 11, the second light source 12, the third light source 13, . . . , the n-th light source In are LEDs, they are provided so as to have the different emission spectra by using different materials for forbidden bands (band gap) used for manufacturing the LEDS.

The light receiver 20 detects the light emitted from the light emitter 10. Specifically, the light receiver 20 is a planar optical sensor panel on which a plurality of optical sensors such as photodiodes are provided and two-dimensionally arranged. Each of the optical sensors produces an output corresponding to illuminance and an irradiation time of the light in an area provided with the optical sensor. The light receiver 20 is provided so as to be capable of outputting two-dimensional data that indicates a degree of detection of the light.

The information processor 30 performs various types of processing related to operations of the detection device 1. The information processor 30 includes a controller 31, a timer 32, and a storage 40.

The controller 31 performs arithmetic processing related to the operations of the detection device 1. Specifically, the controller 31 includes, in the configuration thereof, an arithmetic circuit serving as a central processing unit (CPU), for example, and reads “data stored in the storage 40 and computer software programs (hereinafter, referred to as computer programs and the like), which are not illustrated” and executes and processes them. The timer 32 is a timer circuit provided so as to be capable of measuring passage of time. Hereinafter, the term “processing performed by the information processor 30” refers to processing achieved by the processing performed by the controller 31, unless otherwise noted. For example, the processing for controlling the operation of each component of the detection device 1 performed by the information processor 30 is achieved by the execution and the processing of the computer program and the like corresponding to such operation control by the controller 31, and therefore, falls under the category of processing performed by the information processor 30 that is achieved by the processing performed by the controller 31.

The information processor 30 of the embodiment may be provided as a system-on-chip (SoC), such as an application processor, or may be implemented by a combination of a plurality of circuits.

The storage 40 stores therein various types of data referenced in the processing performed by the controller 31. Specifically, the storage 40 stores therein “correspondence data between the culture medium type and the light source 41” (hereinafter, simply referred to as correspondence data 41) and “colony detection characteristics data 42”. Details of the correspondence data 41 and the colony detection characteristics data 42 will be described later.

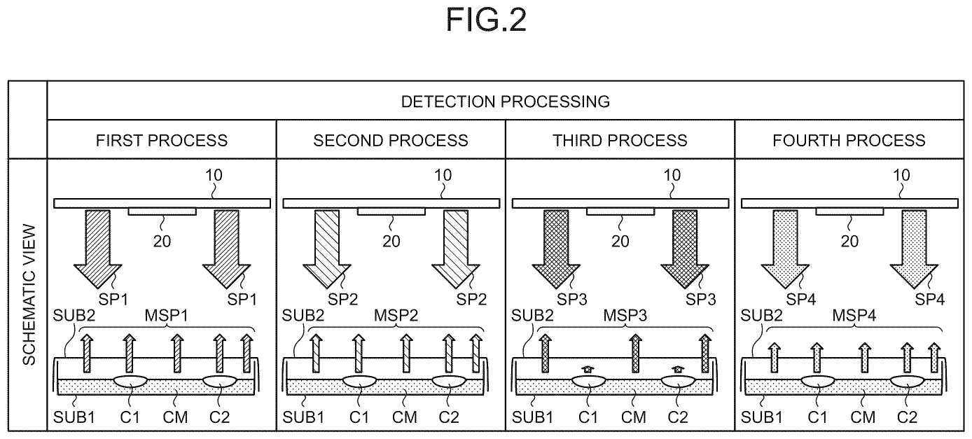

The following describes observation of a culture medium (e.g., agar) performed using the detection device 1, with reference to FIGS. 2 to 8.

FIG. 2 is a diagram schematically illustrating detection processing of the culture medium performed using the detection device 1. Hereinafter, the term “detection processing” refers to detection processing of the culture medium performed using the detection device 1. The detection processing includes a plurality of processes. In each of the processes included in the detection processing, one of the four or more types of the light sources included in the light emitter 10 is turned on and the other types of the light sources are not turned on. The type of the light source that is turned on in each of the processes included in the detection processing is different.

FIG. 2 illustrates an example where the detection processing includes four processes. The four processes are a first process, a second process, a third process, and a fourth process. In FIG. 2, light emitted from the light emitter 10 toward a Petri dish SUB1 in the first process is illustrated as light SP1. Light emitted from the light emitter 10 toward the Petri dish SUB1 in the second process is illustrated as light SP2. Light emitted from the light emitter 10 toward the Petri dish SUB1 in the third process is illustrated as light SP3. Light emitted from the light emitter 10 toward the Petri dish SUB1 in the fourth process is illustrated as light SP4. The light SP1, the light SP2, the light SP3, and the light SP4 are emitted from different light sources.

The Petri dish SUB1 is made of a light-transmitting member and provided, on the upper surface side thereof, with a culture medium CM. The culture medium CM is a culture medium on which colonies can be cultured. The term “colonies” herein refers to colonies formed of biological tissues or microorganisms cultured on the culture medium CM on a configuration such as the Petri dish SUB1 on which the culture medium CM can be provided. More specifically, the Petri dish SUB1 is, for example, a glass Petri dish, but is not limited thereto, and may have another configuration that functions in the same way.

The processes included in the detection processing have in common that the light from the light emitter 10 irradiates the Petri dish SUB1 provided with the culture medium CM and the light receiver 20 detects light affected by the Petri dish SUB1. The light affected by the Petri dish SUB1 refers to reflected light from the Petri dish SUB1 provided with the culture medium CM or transmitted light that has passed through the Petri dish SUB1 provided with the culture medium CM. FIG. 2 illustrates the case where the light affected by the Petri dish SUB1 provided with the culture medium CM is the reflected light.

In the example illustrated in FIG. 2, a lid SUB2 is placed on top of the Petri dish SUB1. The lid SUB2 is a light-transmitting lid placed in order to maintain a more stable culture environment in the Petri dish SUB1 provided with the culture medium CM. The lid SUB2 transmits the light emitted from the light emitter 10 toward the Petri dish SUB1 and the reflected light from the Petri dish SUB1 provided with the culture medium CM.

In FIG. 2, reflected light MSP1 is produced in the first process. Reflected light MSP2 is produced in the second process. Reflected light MSP3 is produced in the third process. Reflected light MSP4 is produced in the fourth process. The reflected light MSP1 is the reflected light produced by the Petri dish SUB1 irradiated with the light SP1. The reflected light MSP2 is the reflected light produced by the Petri dish SUB1 irradiated with the light SP2. The reflected light MSP3 is the reflected light produced by the Petri dish SUB1 irradiated with the light SP3. The reflected light MSP4 is the reflected light produced by the Petri dish SUB1 irradiated with the light SP4.

Colonies C1 and C2 are generated in the culture medium CM illustrated in FIG. 2. Light reflectance in a particular wavelength band differs between the culture medium CM and the colonies C1 and C2. The particular wavelength band is, for example, a part of a reflection spectrum that exhibits an overall tendency of light reflection. In FIG. 2, in the reflected light MSP3 that is the reflected light of the light SP3, components of light reflected by the colonies C1 and C2 are significantly weaker than components of light reflected by the culture medium CM, and this fact is indicated by the lengths of arrows for indicating degrees of reflection in FIG. 2. In contrast, in the reflected light MSP1, MSP2, and MSP4, no significant difference occurs between the components of the light reflected by the culture medium CM and the components of the light reflected by the colonies C1 and C2, unlike the reflected light MSP3. In such reflected light MSP1, MSP2, and MSP4, the relation between the components of the light reflected by the culture medium CM and the components of the light reflected by the colonies C1 and C2 as in the reflected light MSP3 appears in an output of the light receiver 20 that indicates the result of detection of the light.

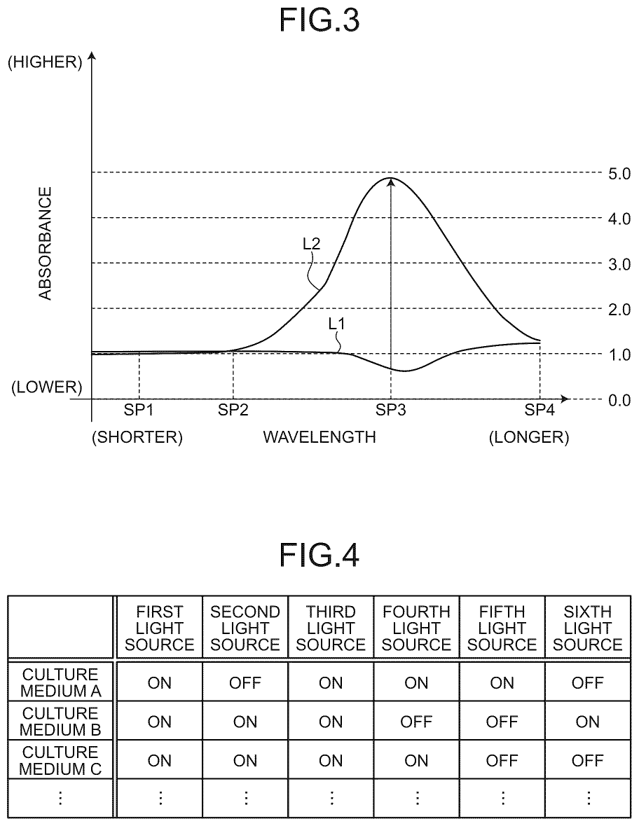

FIG. 3 is a graph illustrating an example of a change in light absorbance between presence and absence of the colonies on the culture medium CM. In the graph in FIG. 3, the horizontal axis indicates the magnitude of wavelength of light, and the vertical axis indicates the light absorbance by an object irradiated with light. Lower light absorbance means that light is more easily reflected, so that the light reflected by the culture medium CM can be more easily detected by the light receiver 20. To explain the relation in the magnitude of wavelength of light among the light SP1, SP2, SP3, and SP4 described with reference to FIG. 2, the light SP1 has a shorter wavelength than the light SP2, SP3, and SP4 in FIG. 3. The light SP2 has a shorter wavelength than the light SP3 and SP4. The light SP3 has a shorter wavelength than the light SP4.

In FIG. 3, graph L1 illustrates the light absorbance by the culture medium CM that is provided on the Petri dish SUB1 and in which the colonies C1 and C2 are not generated. Graph L2 illustrates the light absorbance by the culture medium CM that is provided on the Petri dish SUB1 and in which the colonies C1 and C2 are generated. Graphs L1 and L2 are examples of the reflection spectrum that exhibits the overall tendency of light reflection.

As illustrated in FIG. 3, the light absorbance illustrated by graph L1 is slightly lower at the wavelength of the light SP3 and near that of the light SP3 than at wavelengths of the light from the other light sources (light SP1, SP2, and SP4). However, the differences therebetween are not at a noteworthy level, and the light absorbance is substantially at the same level over the entire wavelength range. In contrast, the light absorbance illustrated by graph L2 is significantly higher at the wavelength of the light SP3 and near that of the light SP3 than at wavelengths of the light from the other light sources (light SP1, SP2, and SP4). That is, the colonies C1 and C2 reduce the reflectance of the light SP3.

Thus, the presence or absence of generation of the colonies such as the colonies C1 and C2 affects the light reflectance in the particular wavelength band included in the reflection spectrum of light reflected by the culture medium CM. This fact indicates that the presence or absence of the colonies such as the colonies C1 and C2 can be determined based on the level of the detection intensity of the light detected by the light receiver 20.

FIG. 4 is a table illustrating exemplary content of the correspondence data 41. In the description with reference to FIG. 4 and subsequent drawings, a case where n=6, that is, the number of types of the light sources is six, as an example. n is not limited to six, and only needs to be four or larger. In the description with reference to FIG. 4 and the subsequent drawings, the six types of the light sources are distinguished by referring to them as a “first light source”, a “second light source”, a “third light source”, a “fourth light source”, a “fifth light source”, and a “sixth light source”. To illustrate the correspondence between FIGS. 1 and 4, the “first light source” is the first light source 11. The “second light source” is the second light source 12. The “third light source” is the third light source 13. The “sixth light source” is the n-th light source In to which n=6 is applied. The “fourth light source” and the “fifth light source” are the light sources that are not illustrated between the third light source 13 and the n-th light source 1n in FIG. 1.

In the embodiment, the “first light source” is a light source that emits red light when turned on. The “second light source” is a light source that emits green light when turned on. The “third light source” is a light source that emits blue light when turned on. The “fourth light source” is a light source that emits cyan light when turned on. The “fifth light source” is a light source that emits magenta light when turned on. The “sixth light source” is a light source that emits yellow light when turned on.

The correspondence data 41 is data in a table format. A record (row) of the correspondence data 41 is individually provided for each type of the culture medium CM (target culture medium). A column of the correspondence data 41 is individually provided for each type of the light source. In a field where one record intersects one column, a parameter is set which indicates lighting (on) or non- lighting (off) of the light source of a type indicated by the one column.

For example, in the example illustrated in FIG. 4, a “culture medium A”, a “culture medium B”, and a “culture medium C” are illustrated as the types of the culture medium CM. In the record of the “culture medium A”, fields intersecting columns of the “first light source”, the “third light source”, the “fourth light source”, and the “fifth light source” are “on” and fields intersecting columns of the “second light source” and the “sixth light source” are “off”. Therefore, it can be read from the record of the “culture medium A” that four types of the “first light source”, the “third light source”, the “fourth light source”, and the “fifth light source” are used when the “culture medium A” is employed as the culture medium CM. It can also be read from the record of the “culture medium A” that two types of the “second light source” and the “sixth light source” are not used when the “culture medium A” is employed as the culture medium CM.

In the record of the “culture medium B”, the fields intersecting the columns of the “first light source”, the “second light source”, the “third light source”, and the “sixth light source” are “on” and the fields intersecting the columns of the “fourth light source” and the “fifth light source” are “off”. In the record of the “culture medium C”, the fields intersecting the columns of the “first light source”, the “second light source”, the “third light source”, and the “fourth light source” are “on” and the fields intersecting the columns of the “fifth light source” and the “sixth light source” are “off”. These records also can be interpreted based on the same concept as that of the parameters (on or off) of the fields in the record of the “culture medium A” described above.

Thus, the correspondence data 41 serves as second data that indicates a relation between the object to be detected (property of the culture medium CM formed on the Petri dish SUB1) irradiated with the light from the light emitter 10 and the light sources that are turned on among the multiple types of the light sources provided in the light emitter 10. By referring to the correspondence data 41, the correspondence between the culture medium CM used in the detection processing and the type of light source as described with reference to FIG. 2 can be identified.

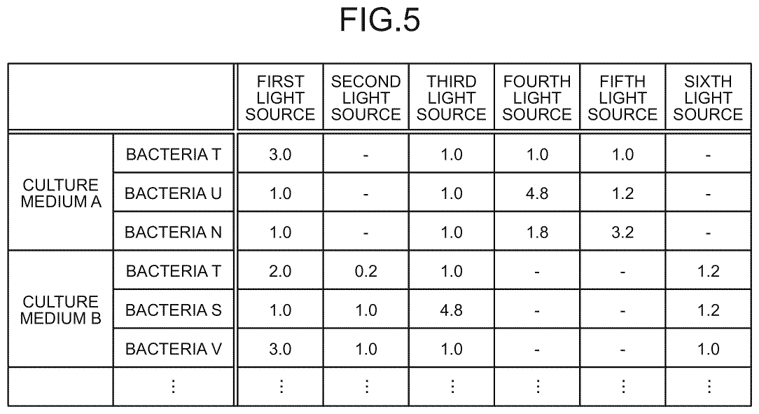

FIG. 5 is a table illustrating exemplary content of the colony detection characteristics data 42. The colony detection characteristics data 42 is data in a table format. The record (row) of the colony detection characteristics data 42 is individually provided for each combination of the type of the culture medium CM (target culture medium) and the type of a culture target (detection target) generated on the culture medium CM of that type. The column of the colony detection characteristics data 42 is individually provided for each type of the light source. In a field where one record intersects one column, a parameter is set that indicates the absorbance of the light from the light source of a type indicated by the one column when the detection target is generated on the target culture medium.

For example, in a record of the combination of the “culture medium A” and “bacteria T”, a parameter “3.0” is set in the field intersecting the column of the “first light source”. This setting “3.0” indicates the absorbance of the light due to the generation of the colonies such as the colonies C1 and C2.

In FIG. 3, numerical values of the absorbance are written on the right side of the graph. The setting “3.0” mentioned above is set based on the relation in which, when a colony of the “bacteria T” is generated in the detection processing of the “culture medium A”, the output of the light receiver 20 obtained by turning on the “first light source” indicates the absorbance of the light corresponding to “3.0” in the graph. Such a parameter is set after being identified in advance through experiments or the like, in which light from the light emitter 10 is emitted to an actual combination of the target culture medium and the culture target and then detected by the light receiver 20.

In the record of the combination of the “culture medium A” and the “bacteria T”, a parameter of “1.0” is set in three fields intersecting three columns of the “third light source”, the “fourth light source” and the “fifth light source” in addition to a parameter of “3.0” of the “first light source” mentioned above. This parameter is set based on the relation in which, when the colony of the “bacteria T” is generated in the detection processing of the “culture medium A”, the output of the light receiver 20 obtained by turning on the “third light source”, the output of the light receiver 20 obtained by turning on the “fourth light source”, and the output of the light receiver 20 obtained by turning on the “fifth light source” each indicate the absorbance of the light corresponding to “1.0” in the graph.

That is, in the detection processing of “cultured medium A”, the “first light source”, the “third light source”, the “fourth light source”, and the “fifth light source” are turned on in separate processes, as described with reference to FIGS. 2 and 4. When the colony of the “bacteria T” is generated in the “culture medium A”, the output of the light receiver 20 is obtained that corresponds to the combination of the absorbance of the light in which the absorbance of the light of the “first light source” is “3.0” and the absorbance of the light of each of the “third light source”, the “fourth light source”, and the “fifth light source” is “1.0”.

The same concept applies to the other records illustrated in FIG. 5. For example, in the record of the combination of the “culture medium A” and “bacteria U”, the “fourth light source” is “4.8”, the “fifth light source” is “1.2”, and the “first light source” and the “third light source” are “1.0”. This setting indicates that, when a colony of the “bacteria U” is generated on the “culture medium A”, the absorbance of the light of the “fourth light source” is “4.8”, the absorbance of the light of the “fifth light source” is “1.2”, and the absorbance of the light of each of the “first light source” and the “third light source” is “1.0”. The record of the combination of the “culture medium A” and “bacteria T” indicates that the output of the light receiver 20 corresponding to the combination of these absorbance values of the light can be obtained.

The detection of the light by the light receiver 20 is performed individually by each of the optical sensors provided in the light receiver 20. In the embodiment, for example, if a predetermined number or more of the optical sensors indicate an output corresponding to a combination of the absorbance of the light corresponding to a record illustrated in FIG. 5, a colony is determined to have been generated by the “culture target” of that record. The predetermined number only needs to be a natural number equal to or larger than one and can be any number within that range.

The relation between the level of the output of the light receiver 20 and the magnitude of the value of the absorbance of the light has been identified in advance by prior experiments or the like, and is provided so that the value of the absorbance of the light can be calculated by the controller 31 by obtaining the output of the light receiver 20.

For each target culture medium, the columns in which the parameters of the absorbance are set in each record in FIG. 5 are limited to the columns of the types of the light sources for which “on” is set in the record in FIG. 4. For example, for the “culture medium A”, the fields intersecting the columns of the “first light source”, the “third light source”, the “fourth light source”, and the “fifth light source” are “on”, and the fields intersecting the columns of the “second light source” and the “sixth light source” are “off” in the record of FIG. 4. Therefore, for the “culture medium A”, the parameters indicating the absorbance of the light are set in the columns of the “first light source”, the “third light source”, the “fourth light source”, and the “fifth light source” in the records in FIG. 5, and hyphens (-) are set in the columns of the “second light source” and the “sixth light source”.

Thus, the colony detection characteristics data 42 serves as first data corresponding to the detection result of light by the optical sensor when the colony is generated in the Petri dish SUB1 provided with the culture medium such as the culture medium CM. By referring to the colony detection characteristics data 42, spectral characteristics when the colony by a specific culture target (detection target) is generated on the specific culture medium CM (target culture medium), can be identified as described with reference to FIG. 3. In other words, the record of the colony detection characteristics data 42 corresponds to the spectral characteristics when the colony is generated as illustrated by graph L2 explained with reference to FIG. 3.

Chromogenic enzyme substrates such as X-Gluc and Magenta-Gal are known as examples of the culture medium CM in which the reflection spectrum significantly changes in response to the generation of the colony as described with reference to FIG. 3. X-Gluc is a compound expressed as C₁₄H₁₃BrClNO₇. X-Gluc develops a blue color when reacting with beta-glucuronidase that is a type of enzyme produced by certain Escherichia coli. Magenta-Gal is an agar medium using sodium lauryl sulfate as a selective agent. Magenta-Gal develops a red color by reacting with beta-galactosidase that is a type of enzyme produced by certain Escherichia coli. The culture medium CM is not limited to X-Gluc and Magenta-Gal, and only needs to be a chromogenic enzyme substrate provided so as to exhibit a change in color when reacting with enzyme produced by certain bacteria, with the same mechanism as that of the relation between X-Gluc and beta-glucuronidase or the like. The culture medium CM may be a composite medium containing a plurality of types of chromogenic enzyme substrates such as X-Gluc and Magenta-Gal according to a plurality of types of culture targets. For such a composite culture medium, it is preferable to turn on light having a wavelength corresponding to a color that undergoes a larger change among changes exhibited by each of the chromogenic enzyme substrates in response to the enzyme. As a result, the change in color corresponding to the generation of the multiple types of the colonies can be detected more reliably on the composite culture medium. For example, for a composite culture medium of X-Gluc and Magenta-Gal, it is preferable to include the following light sources in light sources to be turned on: a light source for emitting blue light and a light source for emitting red light, in consideration of change in color exhibited by X-Gluc and change in color exhibited by Magenta-Gal. Furthermore, since the absorbance of the light having wavelengths near 500 nm tends to increase with the change in color exhibited by Magenta-Gal, a light source corresponding to the wavelengths near 500 nm may be further turned on.

The objects that can be cultured with the chromogenic enzyme substrates (culture target) are not limited to the bacteria and can also be other biological tissues or microorganisms that generate chemicals that react with the chromogenic enzyme substrates. The correspondence data 41 described with reference to FIG. 4 and the colony detection characteristics data 42 described with reference to FIG. 5 correspond to the reaction with the enzyme produced by the specific culture target exhibited by properties of the chromogenic enzyme substrates. In other words, in the detection device 1, the light sources that are turned on in the detection processing are provided so as to be changeable according to the type of the chromogenic enzyme substrate serving as the culture medium CM provided on the Petri dish SUB1 that is irradiated with the light from the light emitter 10.

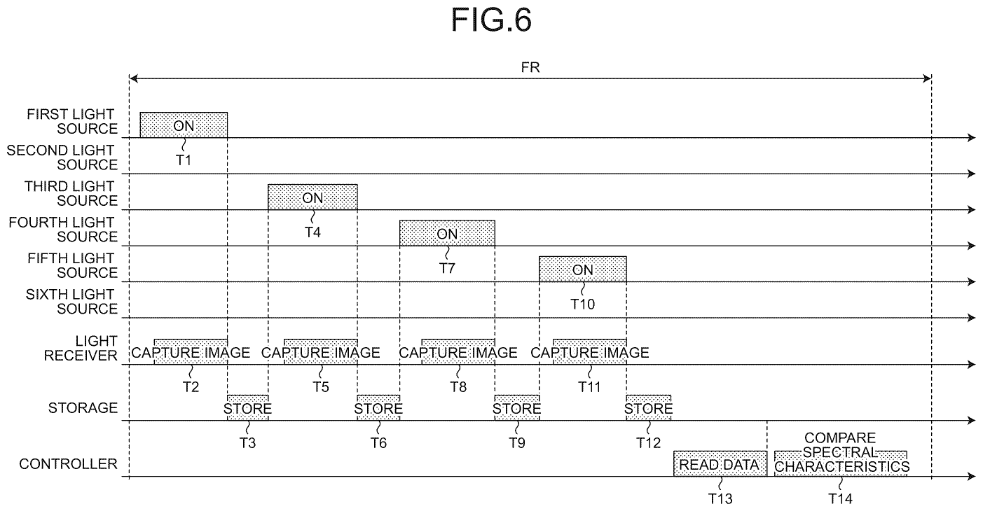

FIG. 6 is a timing diagram illustrating the detection processing. FIG. 6 illustrates an example in which the “culture medium A” described with reference to FIG. 4 is the target culture medium and in which the “first light source”, the “third light source”, the “fourth light source”, and the “fifth light source” are used.

First, the “first light source” is turned on (Step T1). After the process at Step T1 starts, the light receiver 20 detects light (Step T2). The process at Step T2 only needs to start after the process at Step T1 has started. The process at Step T2 only needs to end before the process at Step T1 ends. The process at Step T2 generates and outputs two-dimensional image data in which outputs by the optical sensors of the light receiver 20 are two-dimensionally arranged correspondingly to the arrangement of the optical sensors. In other words, the optical sensors provided in the light receiver 20 serve as what are called imaging pixels. The two-dimensional image data obtained in the process at Step T2 is stored in the storage 40 (Step T3).

In the detection of the light by the light receiver 20 at Step T2 and at Steps T5, T8, and T11 to be described below, the output levels of the optical sensors are reset before the start of the detection of the light, and then the detection of the light starts, at each of the steps. That is, the process at each of the steps is not affected by the output levels produced by previous light detection operations.

Thereafter, in the same manner as the process at Step T1 to the process at Step T3, two-dimensional image data is generated and stored according to the lighting of the light sources other than the “first light source” that are used in the detection processing.

Specifically, the light is detected by the light receiver 20 in the lighting of the “third light source” (Step T4) (Step T5), and the two-dimensional image data generated in the process at Step T5 is stored in the storage 40 (Step T6). The light is detected by the light receiver 20 in the lighting of the “fourth light source” (Step T7) (Step T8), and the two-dimensional image data generated in the process at Step T8 is stored in the storage 40 (Step T9). The light is detected by the light receiver 20 in the lighting of the “fifth light source” (Step T10) (Step T11), and the two-dimensional image data generated in the process at Step T11 is stored in the storage 40 (Step T12).

After the process at Step T12, the two-dimensional image data stored in the storage 40 in the processes at Steps T3, T6, T9, and T12 are read out by the controller 31 (Step T13). Then, spectral characteristics comparison is performed as colony detection determination by referring to the record described with reference to FIG. 5 (Step T14). Specifically, in the same way as in a process at Step S7 to be described later, the spectral characteristics indicated by the read-out data are compared with the colony detection characteristics corresponding to the culture medium.

Specifically, a comparison between the light absorbance indicated by the two-dimensional image data and the light absorbance indicated by the colony detection characteristics data 42 is performed; wherein the light absorbance indicated by the two-dimensional image data means each of the values of the light absorbance indicated by a plurality of pieces of pixel data included in the two-dimensional image data, in other words, each of the values of the light absorbance indicated by the outputs of the optical sensors included in the light receiver 20; and the light absorbance indicated by the colony detection characteristics data 42 means each of the values of the light absorbance indicated by the record corresponding to a combination of the target culture medium and the culture target included in the colony detection characteristics data 42 described with reference to FIG. 5. The record to be compared is limited to the record the target culture medium of which matches the target culture medium of the two-dimensional image data to be compared. In the explanation with reference to FIG. 6, the “culture medium A” is the target culture medium. Therefore, the values of the light absorbance indicated by the pieces of the pixel data included in the two-dimensional image data are compared with the values of the light absorbance indicated by the record in which the target culture medium is the “culture medium A” among the records included in the colony detection characteristics data 42. If the result of the comparison indicates that the predetermined number or more pieces of the pixel data corresponding to the light absorbance indicated by the record included in the colony detection characteristics data 42 are present, it is determined that the colony is generated. In contrast, if less than the predetermined number of pieces of the pixel data corresponding to the light absorbance indicated by the records included in the colony detection characteristics data 42 are present, no colonies are determined to have been generated.

The various processes described with reference to FIG. 6 are performed, for example, by an operation of each component of the detection device 1 under the control of the processing performed by the information processor 30. The processing related to the storing process of the two-dimensional image data, such as the processes at Steps T3, T6, T9, and T12, may be performed by being controlled, for example, mainly by a controller circuit included in the light receiver 20.

Thus, in the embodiment, the controller 31 of the information processor 30 serves as a controller to determine whether the outputs of the optical sensors indicate the generation of the colonies, based on the colony detection characteristics data 42 that serves as the first data mentioned above.

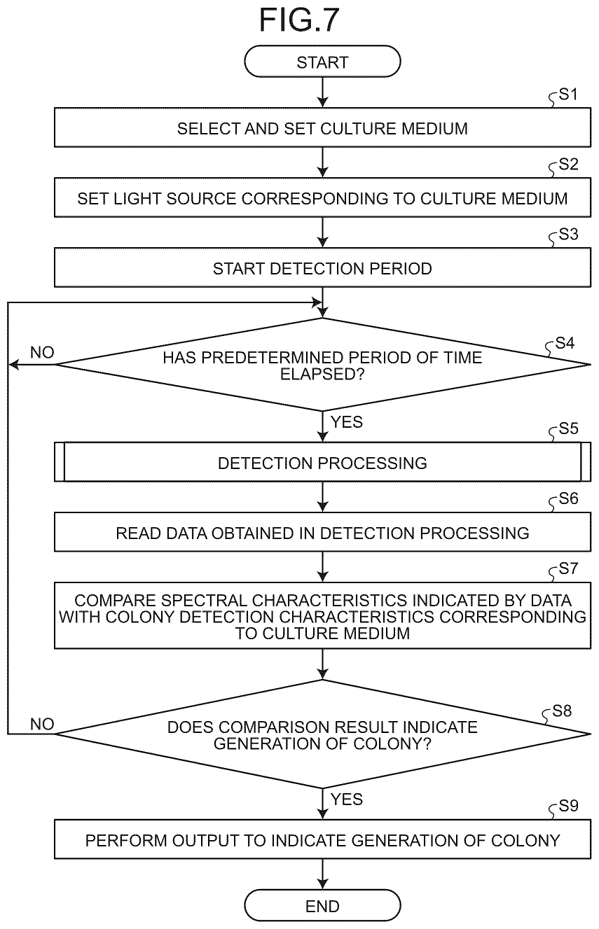

FIG. 7 is a flowchart illustrating processing related to the detection processing. First, the culture medium is selected and set (Step S1). Specifically, any one of the culture media specified as the target culture media in the records of the correspondence data 41 described with reference to FIG. 4 is selected. Then, setting is made to the detection device 1 in accordance with the result of the selection. More specifically, the detection device 1 includes an information input section (not illustrated) and receives input of settings indicating the results of the selection through the input from the information input section. The information input section may be, for example, an input device, such as one or more of a keyboard, a mouse, and a touch panel provided on the detection device 1, or a communication device provided to be capable of receiving data transmitted from an external information processing device, or other configurations.

After the process at Step S1, the information processor 30 performs setting of the light emitter 10 corresponding to the culture medium selected and set in the process at Step S1 (Step S2). Specifically, the controller 31 reads out, from among the records of the correspondence data 41, a record in which the culture medium selected and set in the process at Step S1 is specified as the target culture medium. Then, the controller 31 sets a light source corresponding to a column of the field in the read-out record to which “on” is given, as a light source to be turned on in the detection processing to be described below. A detection period starts (Step S3). Specifically, by using clock output according to timing by the timer 32, the controller 31 starts a process to count a predetermined period of time to manage an execution period of the detection processing to be described below.

After the process at Step S3, the information processor 30 determines whether the predetermined period of time has elapsed (Step S4). Until the predetermined period of time is determined to have elapsed in the process at Step S4, the detection device 1 is kept in a standby state (No at Step S4). If the predetermined period of time is determined to have elapsed in the process at Step S4 (Yes at Step S4), the detection processing is performed (Step S5).

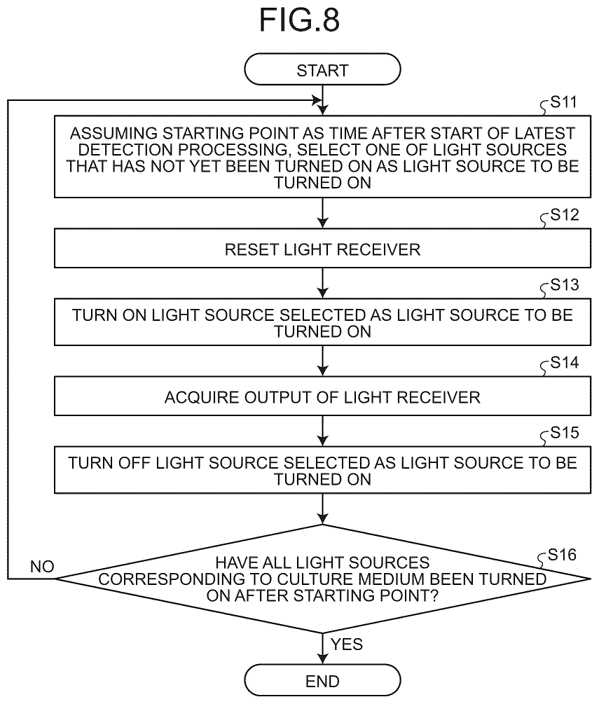

FIG. 8 is a flowchart illustrating the detection processing. First, assuming a starting point as time after the start of the latest detection processing, the information processor 30 selects, as a light source to be turned on, one of the light sources that has not yet been turned on in the process started from the starting point (Step S11). The light receiver is reset (Step S12). Specifically, the potentials of the optical sensors included in the light receiver 20 are reset.

After the processes at Steps S11 and S12, the information processor 30 turns on the light source selected as the light source to be turned on in the latest process of Step S11 (Step S13). After the process at Step S13, the information processor 30 acquires the output of the light receiver 20 (Step S14). The chronological relation between the processes at Steps S13 and S14 is the same as the relation between the processes at Steps T1 and T2 described with reference to FIG. 6. After the process at Step S14, the information processor 30 turns off the light source selected as the light source to be turned on in the latest process at Step S11 (Step S15). After the process at Step S15, the information processor 30 determines whether all the light sources corresponding to the culture medium set and selected in the process at Step S1 have been turned on after the starting point described above (Step S16). Specifically, a determination is made as to whether all light sources to which “on” is given in the record read out in the process at Step S2 are turned on through the process at Step S13. If the process at Step S16 determines that light sources that have not yet been turned on are present among the light sources corresponding to the culture medium (No at Step S16), the process at Step S11 is performed. Thus, the processes from Step S11 to Step S15 are performed for all the light sources to which “on” is given in the record read out in the process at Step S2.

For example, when the “first light source”, the “third light source”, the “fourth light source”, and the “fifth light source” are used, the following four series are performed: a series of processes in which the “first light source” is selected as a light source to be turned on in the process at Step S11; a series of processes in which the “third light source” is selected as a light source to be turned on in the process at Step S11; a series of processes in which the “fourth light source” is selected as a light source to be turned on in the process at Step S11; and a series of processes in which the “fifth light source” is selected as a light source to be turned on in the process at Step S11. The repetition of the series of processes corresponds, for example, to the processes from Step Tl to Step T12 in FIG. 6. The detection processing ends when all the light sources are determined to have been turned on after the starting point described above in the process at Step S16 (Yes at Step S16). As illustrated in the relation between the process at Step S2 and the repetition of the series of processes, in the embodiment, the light sources to be turned on in the detection processing according to the object to be detected to be irradiated with the light from the light emitter 10 are determined based on the correspondence data 41 that serves as the second data.

After the detection processing described with reference to FIG. 8, that is, after the process at Step S5 illustrated in FIG. 7, the data obtained in the detection processing is read out (Step S6). The process at Step S6 corresponds to the process at Step T13 in FIG. 6. After the process at Step S6, the spectral characteristics indicated by the read-out data are compared with the colony detection characteristics corresponding to the culture medium (Step S7). Specifically, the data read out in the process at Step S6, that is, the light absorbance indicated by the pixel data included in the two-dimensional image data described above is compared with the light absorbance indicated by the record of the colony detection characteristics data 42 described with reference to FIG. 5. That is, the process at Step S7 corresponds to the process at Step T14 in FIG. 6.

After the process at Step S7, a determination is made as to whether the result of the comparison performed in the process at Step S7 indicates the generation of a colony (Step S8). Specifically, the information processor 30 determines whether the predetermined number or more pieces of the pixel data corresponding to the light absorbance indicated by the record included in the colony detection characteristics data 42 are present. If the process at Step S8 determines that the result of the comparison does not indicate the generation of a colony (No at Step S8), that is, the number of pieces of the pixel data corresponding to the light absorbance indicated by the record included in the colony detection characteristics data 42 is less than the predetermined number, the process at Step S4 is performed. That is, after the predetermined period of time has elapsed, the processes from Step S5 to Step S8 are performed again. Thus, in the detection processing of the embodiment, the multiple types of the light sources are turned on at different times, and detection is performed by the optical sensors each time any one of the light sources is turned on. The detection processing is performed a plurality of times at intervals of a predetermined period of time. The predetermined period of time is five minutes, for example, but is not limited thereto, and may be any time.

If the process at Step S8 determines that the result of the comparison indicates the generation of a colony (Yes at Step S8), that is, the number of pieces of the pixel data corresponding to the light absorbance indicated by the record included in the colony detection characteristics data 42 is equal to or more than the predetermined number, the information processor 30 performs an output to indicate that a colony has been generated (Step S9). Such an output may be performed by an operation of an output section (not illustrated) provided in the detection device 1, or by transmission of information to the external information processing device via the communication device mentioned above. Examples of the output section include, but are not limited to, a display device such as a display panel, an audio output device such as a speaker, and other output devices for notification.

As described above, according to the embodiment, the detection device 1 includes the light emitter (such as the light emitter 10) provided with multiple types of light sources (such as the first light source 11, the second light source 12, the third light source 13, . . . , the n-th light source In) that emit light in respective different colors, and the light receiver (such as the light receiver 20) that has a detection area in which a plurality of optical sensors are two-dimensionally arranged. In the detection device 1, the detection processing is performed. In the detection processing, the multiple types of the light sources are turned on at different times, and detection is performed by the optical sensors each time any one of the light sources is turned on. In the detection device 1, the light sources are provided such that the light sources to be turned on in the detection processing are changeable depending on the object to be detected that is irradiated with the light from the light emitter (for example, depending on the properties of the chromogenic enzyme substrates that is the culture medium CM provided on the Petri dish SUB1). This configuration allows the optical sensors to detect light in a color corresponding to the object to be detected, color by color. Therefore, unlike in a case where an optical filter decomposes white light, detection results corresponding to colors of light of the light sources can be obtained reliably. That is, in the embodiment, difficulties in distinguishing colors caused by overlapping of intermediate detection intensities of multiple colors, which can occur when the optical filter decomposes white light, cannot occur in principle. Thus, according to the embodiment, colors can be distinguished with higher accuracy.

Since the detection processing is performed a plurality of times at intervals of a predetermined period of time, the determination as to whether a colony has been generated in the culture medium can be performed at intervals of the period of time.

The detection device 1 also includes the storage (such as the storage 40) and the controller (such as the controller 31). The storage stores therein first data (such as the colony detection characteristics data 42) corresponding to the detection result of the light by the optical sensors when the colonies (such as the colonies C1 and C2) are generated in the object to be detected (such as the Petri dish SUB1) provided with the culture medium (such as the culture medium CM). The controller determines, based on the first data, whether the outputs of the optical sensors obtained in the detection processing indicate the generation of the colonies. Thereby, the determination as to whether colonies have been generated in the culture medium can be automatically performed based on the outputs of the optical sensors obtained in the detection processing.

The multiple types of the light sources provided in the light emitter (such as the light emitter 10) includes the light source that emits the red light, the light source that emits the green light, the light source that emits the blue light, and one or more light sources that emit light in a color or colors different from red, green, and blue. Thereby, a detection result of a specific spectral pattern corresponding to the color or colors different from red, green, and blue can be obtained in addition to the three primary colors of what are called RGB colors. Therefore, colors different from red, green, and blue can be identified with higher precision.

The multiple types of the light sources provided in the light emitter (such as the light emitter 10) includes one or more of the light source that emits cyan light, the light source that emits magenta light, and the light source that emits yellow light. Thereby, the colors emitted by the light sources included in the light emitter can be distinguished with higher accuracy. In particular, since the colors are cyan, magenta, and yellow, complementary colors of what are called RGB colors can be distinguished with higher accuracy.

In the configuration where the storage (such as the storage 40) stores therein the second data (such as the correspondence data 41) indicating the relation between the object to be detected (for example, the properties of the chromogenic enzyme substrates that is the culture medium CM provided on the Petri dish SUB1) irradiated with the light from the light emitter (such as the light emitter 10) and the light sources that are turned on among the multiple types of the light sources, the light sources to be turned on in the detection processing according to the object to be detected to be irradiated with the light from the light emitter are determined based on the second data. Thereby, the light sources appropriate for properties of the object to be detected can be used more easily for the detection processing. In other words, the light sources that emit light corresponding to colors to be distinguished can be turned on in the detection processing.

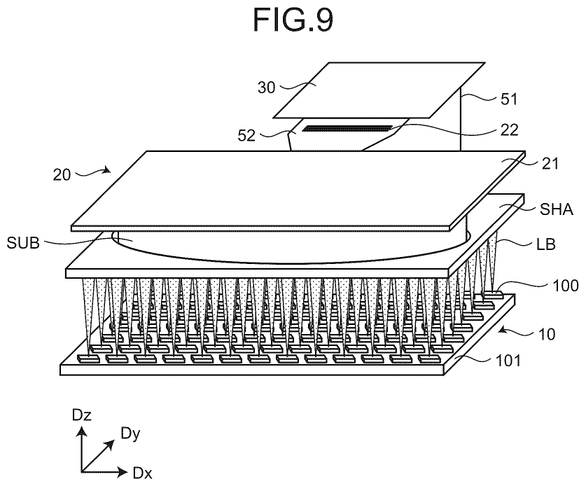

The positional relation between the light emitter 10 and the light receiver 20 is not limited to the relation described with reference to FIG. 2. The light emitted from the light emitter 10 and affected by the object to be detected is not limited to the reflected light described above, and may be light transmitted through the object to be detected. The following describes a configuration example of the detection device when the light affected by the object to be detected is the light transmitted through the object to be detected, with reference to FIG. 9.

FIG. 9 is a schematic view illustrating the configuration example of the detection device when the light affected by the object to be detected is the light transmitted through the object to be detected. As illustrated in FIG. 9, in the detection device, a Petri dish SUB is placed between the light emitter 10 and the light receiver 20. The Petri dish SUB has a configuration including, for example, the Petri dish SUB1 and the lid SUB2 described above. In the Petri dish SUB, a culture medium such as the culture medium CM (refer to FIG. 2) is formed, and colonies such as the colonies C1 and C2 (refer to FIG. 2) can be generated. The Petri dish SUB is placed on a placement member SHA. The placement member SHA serves as a member where the Petri dish SUB can be placed such that the Petri dish SUB is interposed between the light emitter 10 and the light receiver 20. The placement member SHA has a configuration including a placement portion on which the Petri dish SUB is placed and that is made of a light-transmitting member and a peripheral portion that is located on the outer peripheral side of the placement portion and is made of a light-blocking member. As a specific example, the light-transmitting member is made of glass or a colorless resin, and the light-blocking member is made of a black resin.

Wiring 51 illustrated in FIG. 9 is wiring that couples the light emitter 10 to the information processor 30. Wiring 52 is wiring that couples the light receiver 20 to the information processor 30. A circuit 22 is the controller circuit of the light receiver 20. In FIG. 9, light sources 100 provided in the light emitter 10 each include the first light source 11, the second light source 12, the third light source 13, . . . , the n-th light source In. A substrate 101 is a substrate of the light emitter 10 on which the light sources 100 are two-dimensionally arranged. A substrate 21 is a substrate of the light receiver 20 on which the optical sensors are two-dimensionally arranged. When light affected by the Petri dish SUB is light transmitted through the Petri dish SUB in the same way as the example described with reference to FIG. 9, it is preferable that the culture medium CM provided in the Petri dish SUB does not have a perfect light-blocking property but exhibits a degree of light transmissibility that changes depending on presence or absence the colonies and the thickness of the colonies.

In the description with reference to FIG. 5, the parameter as a value indicating the light absorbance is a fixed value, but is not limited to the fixed value. For example, the parameter may have a range of the light absorbance that can be produced depending on the combination of the target culture medium and the culture target. That is, a colony by the target culture medium in a certain record may be determined to have been generated if the output of the light receiver 20 obtained in the process corresponding to the combination of the target culture medium and the type of the light source set in the certain record is within the range of the light absorbance set in the field corresponding to the combination in the record. Thus, the first data (such as the correspondence data 41) indicates the range of output that can be produced when the output of the optical sensor obtained in the detection processing described above indicates the generation of the colonies; and when the output of the optical sensor obtained in the detection processing is within this range, the controller (such as the controller 31) determines that colonies are generated. Thereby, colors can be more accurately distinguished under more flexible determination conditions.

In the description with reference to FIG. 4 and the subsequent drawings, n=6 and the number of types of the light sources turned on in the detection processing is four. However, these numbers are only exemplary and can be changed as appropriate. Specifically, the number of types of the light sources that are turned on in the detection processing only needs to be four or larger. Although the light sources to be turned on in the detection process may be determined according to the properties of the chromogenic enzyme substrates, four or more light sources are preferably turned on.

The colors of light used in the detection device 1 are not limited to red, green, blue, cyan, magenta, and yellow listed above, and may be other colors of light depending on the properties of the chromogenic enzyme substrates.

Other operational advantages accruing from the aspects described in the present embodiment that are obvious from the description herein, or that are conceivable as appropriate by those skilled in the art will naturally be understood as accruing from the present disclosure.

What is claimed is:

1. A detection device comprising:
a light emitter provided with multiple types of light sources configured to emit light in different colors from one another; and
a light receiver in which a plurality of optical sensors are two-dimensionally arranged, wherein
the detection device is configured to perform detection processing,
in the detection processing, the multiple types of the light sources are turned on at different times from one another, and detection is performed by the optical sensors each time any one of the light sources is turned on, and
the light sources are provided such that the light sources to be turned on in the detection processing are changeable depending on an object to be detected that is irradiated with the light from the light emitter.

2. The detection device according to claim 1, wherein the detection processing is performed a plurality of times at intervals of a predetermined period of time.

3. The detection device according to claim 1, comprising:
a storage configured to store first data corresponding to a detection result of light by the optical sensors when a colony is generated in the object to be detected provided with a culture medium; and
a controller configured to determine, based on the first data, whether outputs of the optical sensors obtained in the detection processing indicate generation of the colony.

4. The detection device according to claim 1, wherein the multiple types of the light sources comprise:
a light source configured to emit red light;
a light source configured to emit green light;
a light source configured to emit blue light; and
one or more light sources configured to emit light in a color or colors different from red, green, and blue.

5. The detection device according to claim 4, wherein the one or more light sources comprise one or more of a light source configured to emit cyan light, a light source configured to emit magenta light, and a light source configured to emit yellow light.

6. The detection device according to claim 3, wherein
the storage is configured to store second data that indicates a relation between the object to be detected irradiated with the light from the light emitter and the light sources to be turned on among the multiple types of the light sources, and
light sources to be turned on in the detection processing according to the object to be detected to be irradiated with the light from the light emitter are determined based on the second data.

7. The detection device according to claim 3, wherein
the first data indicates a range of the output that is producible when the output of the optical sensor obtained in the detection processing indicates the generation of the colony, and
the controller is configured to determine that the colony is generated when the output of the optical sensor obtained in the detection processing is within the range.
